(19) United States
(12) Patent Application Publication
(10) Pub. No.: US 2019/0117533 A1
(43) Pub. Date: Apr. 25, 2019

(54) SUNSCREEN COMPOSITION CONTAINING SURFACE-MODIFIED CERIUM OXIDE PARTICLES

(71) Applicant: SOULBRAIN CO., LTD., Seongnam-si (KR)

(72) Inventors: SeungHyun LEE, Seongnam-si (KR); Seok Joo KIM, Seongnam-si (KR)

(21) Appl. No.: 16/164,836

(22) Filed: Oct. 19, 2018

(30) Foreign Application Priority Data
Oct. 20, 2017 (KR) 10-2017-0136577

(51) Int. Cl. A61K 8/19; A61Q 17/04; A61K 8/58

(52) U.S. Cl. CPC A61K 8/19; A61K 8/585; A61Q 17/04

(57) ABSTRACT

The present invention provides a sunscreen composition comprising cerium oxide (CeO2) particles that are surface-modified with silicone, wherein the silicone is methicone, a methicone derivative, or an alkyl silane. The sunscreen composition according to an embodiment of the present invention is easy to be prepared into a formulation applicable to cosmetics and may have a high physical ultraviolet protection effect to block ultraviolet rays in a wide range of wavelengths with an excellent sun protection factor (SPF). In addition, the sunscreen composition according to an embodiment of the present invention has a skin-like color in view of the color tone of the composition and does not cause white cast even when applied to the skin, and thus the composition can be effectively used in producing cosmetics for UV protection that aim to express natural color.

### CROSS-REFERENCE TO RELATED APPLICATIONS

This application claims priority under 35 U.S.C. § 119 to Korean Patent Application No. 10-2017-0136577, filed on Oct. 20, 2017, in the Korean Intellectual Property Office, the disclosure of which is incorporated herein by reference in its entirety.

### TECHNICAL FIELD

The following disclosure relates to a sunscreen composition containing cerium oxide particles that are surface-modified with silicone.

### BACKGROUND

Since cosmetics containing sunscreen were developed for the first time in the United States in 1928, demand for sunscreens has been steadily increasing. Sunscreen aims to prevent skin cancer, sunburn, sunlight aging, and the like, caused by ultraviolet rays. Recently, there is an increased interest in preventing sunlight aging through blocking of the ultraviolet rays corresponding to UVA1 and UVA2 wavelengths for the purpose of beauty. In addition to sunscreen, UV protection function is also added to most formulations such as BB cream, CC cream, sun cushion, sun spray, sun stick, and the like.

In order to block ultraviolet rays, sunscreen is added to the formulations, and the added sunscreen can be divided into organic sunscreens and inorganic sunscreens. Organic sunscreen is representatively a chemical sunscreen that converts light into heat, while inorganic sunscreen is representatively a physical sunscreen that reflects, scatters, and absorbs light. Unlike basic cosmetics, sunscreen is mainly used to attenuate ultraviolet rays at an upper part of the epidermis, i.e., at the outermost part of the skin. However, organic sunscreens such as avobenzone are likely to penetrate the skin due to their small molecular size. Organic sunscreen has advantages of low white cast and a wide range of absorption wavelengths, but has disadvantages of causing skin trouble or side effects such as a burning sensation in the eyes when applied to areas near the eyes in the case of having a sensitivity. Meanwhile, inorganic sunscreen is relatively advantageous in safety and has good sun protection power, but problems such as white cast may occur since it is a white pigment having a high refractive index. Due to the nature-friendly trend of cosmetic materials in recent years, in Korea, there has been a high preference for UV protection products in the category of ‘inorganic sunscreen’ formulations which are composed of only an inorganic sunscreen as a functional component.

Titanium dioxide (Tio₂) and zinc oxide (ZnO) are used as inorganic sunscreens, but there are various disadvantages to their use. First, the energy bandgaps of titanium dioxide and zinc oxide are 3.0 eV and 3.2 eV, respectively, which are advantageous for absorption of UVB and UVA2, and thus it is not possible to absorb UVA1, the intermediate wavelength, with titanium dioxide and zinc oxide alone. Second, the refraction indexes of titanium dioxide and zinc oxide are high at 2.7 and 2.2, respectively, and thus a white cast phenomenon in which white color appears when applied to the skin may appear prominently. Third, titanium dioxide and zinc oxide may cause component denaturation and pigmentation of the formulation due to their large photocatalytic power which decomposes or denatures organic material, especially pigment, when exposed to light energy. In particular, when the photocatalytic power is large, it is necessary for a surface to be covered with a second material for safety reasons. In the case of titanium dioxide, the surface is covered with aluminum oxide (Al₂O₃) or silicon dioxide (SiO₂) up to at an amount of 20 parts per weight or more. However, when the surface is covered with aluminum oxide and silicon dioxide, there are disadvantages in that the powder texture is heavy, and the composition is not softly applied, thus resulting in a hard and dry feeling of use. Accordingly, there is a need to develop a sunscreen composition capable of compensating for the above-described disadvantages.

Therefore, the present inventors studied sunscreen and found that when cerium oxide was surface-modified with silicone and used as a sunscreen composition, the composition was easily applicable to cosmetics and the dispersibility thereof increased, thus resulting in enhancement of the physical UV protection effect and improvement in the areas of stability of the formulation, skin irritation relief, texture, spreadability, and the feeling of use, thus completing the present invention.

### SUMMARY

An embodiment of the present invention is directed to providing an ultraviolet (UV) sunscreen composition capable of absorbing UVA1 and having outstanding stability and feeling of use such as texture, spreadability, and the like.

Another embodiment of the present invention is directed to providing a method for preparing the sunscreen composition described above.

The present invention provides a sunscreen composition comprising cerium oxide (CeO₂) particles that are surface-modified with silicone, wherein the silicone is methicone, a methicone derivative, or an alkyl silane.

Here, the methicone and methicone derivative may have a degree of polymerization of 3 to 50, the silicone may be dimethicone, hydrogen dimethicone, triethoxysilylethyl polydimethylsiloxyethyl hexyl dimethicone, dimethicone copolyol, dimethicone crosspolymer, cyclomethicone, trimethicone, amodimethicone, dimethiconol, cyclomethicone, phenyl trimethicone, cetyl dimethicone, methylphenyl polysiloxane, cyclohexasiloxane, cyclomethasiloxane, or triethoxycaprylylsilane.

Further, the silicone may be a compound represented by Chemical Formula 1 below:

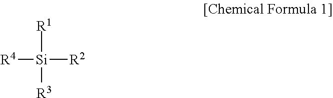

In Chemical Formula 1, R¹, R², R³ and R⁴ are the same as defined in this specification.

The coating density of the surface modification may be 10 to 100% based on the surface area of the cerium oxide particle, the cerium oxide particle may be doped with one or more selected from a group consisting of Al, Ti, Zn, Zr and Ca, the doping amount may be 0.1 to 10 wt % based on the cerium oxide particle, and the cerium oxide particle may have a particle size of 0.05 to 1 μm.

In addition, the content of the cerium oxide particles that are surface-modified with silicone may be 3 to 30 wt % based on the total sunscreen composition.

Further, to achieve the above-described technological goals, the present invention provides a method for preparing a sunscreen composition, comprising a step of adding cerium oxide particles and silicone to an organic solvent and stirring the obtained solution, a step for drying the stirred solution to obtain cerium oxide particles that are surface-modified with silicone, and a step of mixing purified water with the cerium oxide particles surface-modified with silicone and one or more substances selected from a group consisting of silicone oil, fibers, emulsifiers, moisturizers, and plasticizers.

### BRIEF DESCRIPTION OF THE DRAWINGS

FIG. 1 shows images obtained after applying each of the sunscreen compositions prepared in Example 1, Example 2, Comparative Example 2, and Comparative Example 3 onto the skin once.

FIG. 2 shows images obtained after rubbing each of the sunscreen compositions prepared in Example 1, Example 2, Comparative Example 2, and Comparative Example 3 onto the skin several times to be uniformly spread.

FIGS. 3, 4, 5, and 6 are graphs showing sun protection factor (SPF) spectra of the sunscreen compositions prepared in Example 1, Example 2, Comparative Example 2, and Comparative Example 3, respectively.

### DETAILED DESCRIPTION OF EMBODIMENTS

Hereinafter, a detailed description of the present invention is provided.

It should be understood, however, that the following description is an example only, and the present invention is only limited by the scope of the following claims. In addition, the terms used in the invention are only used to describe certain embodiments, and they are not intended to limit the scope of the invention. The expression of singular or plural words is only made in context of grammar, and can include both meanings. Unless specifically stated otherwise, expressions such as “including” or “comprising” do not indicate the exclusion of all other components, and additional components may also be included.

An aspect of the present invention provides a sunscreen composition including cerium oxide (CeO₂) particles that are surface-modified with silicone, wherein the silicone is methicone, a methicone derivative, or an alkyl silane.

Here, the cerium oxide particles may be produced from a cerium precursor such as cerium hydroxide, cerium carbonate, cerium nitrate, cerium chloride, ammonium cerium nitrate, or the like. Any cerium oxide particles produced by a conventional method for producing cerium oxide may be used without any particular limitation.

In addition, the cerium oxide particles may have a plate shape, a flake shape, a spherical shape, or the like, and there is no particular limitation on the shape of the cerium oxide particles.

The cerium oxide particles may have a particle size of 0.03 to 3 μm, 0.04 to 2 μm, and more preferably 0.05 to 1 μm. When the particle size of the cerium oxide particles is less than 0.05 μm, ultraviolet rays may not be sufficiently absorbed due to the excessively small size of the surface-modified cerium oxide particles. When the particle size of the cerium oxide particles exceeds 1 μm, surface-modification with silicone may not be properly achieved due to the excessively large size of the surface-modified cerium oxide particles, leading to a low degree of emulsification of the cerium oxide particles, and thus it may be difficult to produce a proper cosmetic formulation.

In addition, the cerium oxide particles may be doped with one or more metals selected from a group consisting of Al, Ti, Zn, Zr, and Ca. Here, the amount of the metal used for doping may be 0 to 20 wt %, 0 to 15 wt %, and more preferably 0 to 10 wt %, based on the cerium oxide particles. If the doping amount exceeds 10 wt %, the coating density may increase excessively due to excessive doping, thereby increasing the white cast phenomenon. The metal doping can reduce loss of the UV protection effect caused by a photocatalytic reaction or increase in the refractive index of the cerium particles, and the like, and can increase the silicon coating density of the cerium oxide particles, thereby resulting in improvement of dispersibility, and thus it is possible to impart dispersion stability of the formulation and improve the spreadability when applied to the skin.

The cerium oxide particles may be included in the sunscreen composition to absorb the wavelength of the UVA1 region, which is the UV intermediate wavelength region, and may thereby act to broaden the UV absorption region of the sunscreen composition.

The methicone and methicone derivative may have a degree of polymerization of 1 to 200, 2 to 100, and more preferably 3 to 50. When the degree of polymerization is 3 or less, it is difficult to exhibit hydrophobicity, thereby resulting in deterioration of the spreadability, and when the degree of polymerization exceeds 50, a phenomenon such as stickiness, or the like, may occur due to excessively high hydrophobicity, thereby resulting in deterioration of the feeling of use of the composition.

In addition, the silicone may be dimethicone, hydrogen dimethicone, triethoxysilylethyl polydimethylsiloxyethyl hexyl dimethicone, dimethicone copolyol, dimethicone crosspolymer, cyclomethicone, trimethicone, amodimethicone, dimethiconol, cyclomethicone, phenyl trimethicone, cetyl dimethicone, methylphenyl polysiloxane, cyclohexasiloxane, cyclomethasiloxane, or triethoxycaprylylsilane, and the like.

Further, the silicon may be a compound represented by Chemical Formula 1 below.

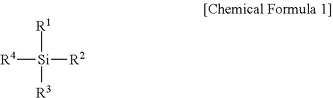

Here, in Chemical Formula 1, R¹, R², R³ and R⁴ may be each, independently: hydrogen; a halogen; a linear or branched C₁-C₁₂ alkyl; a linear or branched C₁-C₁₂ alkyl substituted with one or more substituents selected from a group consisting of an amide group, a hydroxyl group, and a halogen; a linear or branched C₁-C₁₂ alkoxy; a linear or branched C₁-C₁₂ alkoxy substituted with one or more substituents selected from a group consisting of an amide group, a hydroxyl group, and a halogen; a C₃-C₁₂ cycloalkyl; or a C₃-C₁₂ cycloalkyl substituted with one or more substituents selected from a group consisting of an amide group, a hydroxyl group, and a halogen, provided that R¹, R², R³ and R⁴ are not all hydrogen.

Further, in Chemical Formula 1, R¹, R², R³ and R⁴ may be each, independently: hydrogen; a halogen; a linear or branched C₁-C₁₀ alkyl; a linear or branched C₁-C₁₀ alkyl substituted with one or more substituents selected from a group consisting of an amide group, a hydroxyl group, and a halogen; a linear or branched C₁-C₈ alkoxy; a linear or branched C₁-C₈ alkoxy substituted with one or more substituents selected from a group consisting of an amide group, a hydroxyl group, and a halogen; a C₃-C₈ cycloalkyl; or a C₃-C₈ cycloalkyl substituted with one or more substituents selected from a group consisting of an amide group, a hydroxyl group, and a halogen, provided that R¹, R², R³ and R⁴ are not all hydrogen.

Further, in Chemical Formula 1, R¹, R², R³ and R⁴ may be each, independently: hydrogen; a halogen; a linear or branched C₁-C₁₀ alkyl; a linear or branched C₁-C₁₀ alkyl substituted with one or more substituents selected from a group consisting of an amide group, a hydroxyl group, and a halogen; a linear or branched C₁-C₈ alkoxy; or a linear or branched C₁-C₈ alkoxy substituted with one or more substituents selected from a group consisting of an amide group, a hydroxyl group, and a halogen, provided that R¹, R², R³ and R⁴ are not all hydrogen.

In addition, in Chemical Formula 1, R¹ may be a linear or branched C₁-C₁₀ alkyl; or a linear or branched C₁-C₁₀ alkyl substituted with one or more substituents selected from a group consisting of an amide group, a hydroxyl group, and a halogen; and R², R³, and R⁴ may be each, independently, a linear or branched C₁-C₈ alkoxy; or a linear or branched C₁-C₈ alkoxy substituted with one or more substituents selected from a group consisting of an amide group, a hydroxyl group, and a halogen.

Further, the coating density of the surface modification may be 10 to 100% based on the surface area of the cerium oxide particles. When the coating density is less than 10%, the surface modification of the cerium oxide particle is not properly performed, and thus the dispersibility and emulsifying property of the cerium oxide may be lowered, thereby resulting in low stability of the formulation.

The cerium oxide particles surface-modified with silicone may be obtained by surface-modifying existing hydrophobic cerium oxide particles with the above-described silicone substance, thereby allowing the cerium oxide particles to have excellent dispersibility and emulsifiability. Thus, it is possible to produce the sunscreen composition in a form such as a cream or lotion, which are the representative formulations of sunscreen, and to enhance the feeling of use such as stability, spreadability, and the like, of the formulation.

The content of the cerium oxide particles surface-modified with silicone may be from 5 to 50 wt %, based on the total sunscreen composition.

When the content of the cerium oxide particles surface-modified with silicone is less than 5 wt %, the ultraviolet absorption power may be lowered due to the excessively small content of the cerium oxide particles contained in the sunscreen, thus resulting in deterioration of the UV protection effect. When the content of the cerium oxide particles surface-modified with silicone exceeds 50 wt %, the stability of the formulation of the composition may be lowered due to the excessively large content of the cerium oxide particles contained in the sunscreen, thus resulting in deterioration of the feeling of use such as spreadability, and the like.

The sunscreen composition may further include one or more UV protection particles such as titanium oxide particles, zinc oxide particles, or the like.

Here, any content ratio of the UV protection particles, such as the titanium oxide particles and the zinc oxide particles, can be used without particular limitation depending on the purpose and usage to be applied, within the range in which the effects of the present invention are not impaired.

Further, the sunscreen composition may further include compounds such as an organic solvent, silicone oil, a fiber, an emulsifier, a moisturizer, a plasticizer, purified water, or the like.

Here, the organic solvent may be methanol, ethanol, isopropanol, ethyl carbonate, ethyl acetate, benzyl alcohol, benzyl benzoate, propylene glycol, 1,3-butyl glycol oil, glycerol aliphatic ester, polyethylene glycol, sorbitan fatty acid ester, heptane, and the like.

Further, the silicone oil may be dimethicone, cetyl dimethicone, cyclopentasiloxane, cyclohexasiloxane, phenyl trimethicone, stearyl dimethicone, and the like, and the fiber may be VGL silk, and the like.

The emulsifier may be PEG silicone emulsifier, non-ionic W/O emulsifier, polysorbate 60, lanolin, sorbitan olivate, Carnauba wax, olive liquid, lecithin, stearic acid, borax, cetostearyl alcohol, solubilizer, cetyl alcohol, Polysorbate 80, sorbitan stearate, polyoxyetylene phytosterol, hydrogenated soybean phospholipid, and the like. The emulsifier allows each component of the sunscreen composition according to the present invention to be emulsified.

The moisturizer may be polyols such as 1,2-hexanediol, glycerin, propylene glycol, butylene glycol, polyethylene glycol, sorbitol, trehalose, and the like; natural moisturizing factors (NMFs) such as amino acids, urea, lactate, PCA-Na, and the like; and polymeric moisturizers such as hyaluronate, chondroitin sulfate, hydrolyzed collagen, and the like. The moisturizer may increase the moisturizing power of the sunscreen composition according to the present invention and simultaneously act as a preservative.

The plasticizer may be dipropylene glycol (DPG), and the like.

In addition to the above-described components, the sunscreen composition according to the present invention may be appropriately blended with components that are mixed in general cosmetic compositions such as oil, wax, surfactants, thickeners, pigments, cosmetic additives, powders, saccharides, antioxidants, buffers, various extracts, stabilizers, preservatives, fragrances, and the like, within the range in which the effects of the present invention are not impaired.

Here, the oil may be vegetable oils such as evening primrose oil, rosehip oil, castor oil, olive oil, and the like, animal oils such as mink oil, squalane, and the like, mineral oils such as liquid paraffin, vaseline, and the like, or synthetic oils such as silicone oil, isopropyl myristate oil, and the like.

The wax may be a vegetable wax such as carnauba wax, candelilla wax, jojoba oil, and the like, and animal wax such as beeswax, lanolin, and the like.

The surfactant may be an anionic surfactant, a cationic surfactant, an amphoteric surfactant, a non-ionic surfactant, and the like.

In addition, the thickener may be, for example, a water-soluble polymer.

Examples of the water-soluble polymer may include plant-based (polysaccharide-based) natural polymers such as guar gum, locust bean gum, quince seed, carrageenan, galactan, gum arabic, tragacanth gum, pectin, mannan, starch, and the like; microbial (polysaccharide-based) natural polymers such as xanthan gum, dextran, succinol glucan, curdlan, hyaluronic acid, and the like; animal-based (protein-based) natural polymers such as gelatin, casein, albumin, collagen, and the like; cellulose-based semisynthetic polymers such as methyl cellulose, ethyl cellulose, hydroxyethyl cellulose, hydroxypropyl cellulose, carboxymethyl cellulose, methylhydroxypropyl cellulose, and the like; starch-based semisynthetic polymers such as soluble starch, carboxymethyl starch, methyl starch, and the like; alginic acid-based semisynthetic polymers such as alginic acid propylene glycol ester, alginate, and the like; other polysaccharide-based derivative semisynthetic polymers; vinyl-based synthetic polymers such as polyvinyl alcohol, polyvinylpyrrolidone, polyvinylmethylether, carboxyvinyl polymer, sodium polyacrylate, and the like; other synthetic polymers such as polyethylene oxide, ethylene oxide, propylene oxide block copolymers, and the like; and inorganic materials such as bentonite, laponite, microdispersed silicon, colloidal alumina, and the like.

The pigment may be, for example, a synthetic pigment or a natural pigment, wherein the synthetic pigment may be: water-soluble/oil-soluble pigments such as FD&C Yellow No. 6, FD&C Red No. 4, and the like; inorganic pigments such as iron oxide, ultramarine, and the like; organic pigments such as D&C Red No. 30, D&C Red No. 36, and the like; and lakes such as FD&C Yellow No. 6 Al lake, and the like; and the natural pigment may be: carotenoid-based pigments such as β-carotene, β-apo-8-carotenal, rilopine, capsanthin, bixin, crocin, canthaxanthin, and the like; flavonoid-based pigments such as shisonin, lamanine, ninocyanine, carthamin, safrole yellow, rutin, quercetin, cocoa pigment, and the like; flavin-based pigments such as riboflavin, and the like, quinone-based pigments such as laccaic acid, carminic acid (cochineal), kermesic acid, alizanine, shikonin, alkannin, nikino chrome, and the like; porphyrin-based pigments such as chromophil, hemoglobin, and the like; diketone-based pigments such as curcumin (turmeric), or the like; and a betacyanine-based pigment such as betanine, and the like.

The cosmetic additive may be, for example, a vitamin, a plant extract, or an animal extract, wherein the vitamin may be retinol (vitamin A), tocopherol (vitamin E), ascorbic acid (vitamin X), and the like; the plant extract may be menthol (peppermint), azulene (chamomile), allantoin (wheat), caffeine (coffee), licorice extract, cinnamon extract, green tea extract, lavender extract, lemon extract, and the like; and the animal extract may be placenta (placenta of cattle), royal jelly (bee secretion), snail extract (mucus secretion), and the like.

Another aspect of the present invention provides a method for preparing a sunscreen composition, including steps of: adding cerium oxide particles and silicone to distilled water and stirring the obtained solution; drying the stirred solution to obtain cerium oxide particles surface-modified with silicone; and mixing the obtained cerium oxide particles surface-modified with silicone and one or more substances selected from a group consisting of silicone oil, fibers, emulsifiers, moisturizers, and plasticizers, in purified water.

Hereinafter, each step of the method for preparing a sunscreen composition will be described in detail.

The method for preparing a sunscreen composition according to an aspect of the present invention includes a method for producing cerium oxide particles surface-modified with silicone, which includes a step of adding cerium oxide particles and silicone to distilled water and stirring the obtained solution; and a step of drying the stirred solution to obtain cerium oxide particles surface-modified with silicone.

First, the step of adding cerium oxide particles and silicone to distilled water and stirring the obtained solution is performed in order to add cerium oxide particles to distilled water, disperse the cerium oxide particles, add silicone thereto, and react the dispersed cerium oxide particles with the silicone, thereby surface-modifying the cerium oxide with silicone.

Here, the types, contents, and characteristics of the cerium oxide particles and silicone are the same as described above.

The stirring temperature may be 20 to 50° C., and more preferably 25 to 35° C.; the stirring time may be 10 to 180 minutes, and more preferably 30 to 60 minutes; and the stirring speed may be 100 rpm to 2,000 rpm, and more preferably 500 rpm to 1,000 rpm.

Next, the step of drying the stirred solution to obtain cerium oxide particles surface-modified with silicone is performed in order to dry the cerium oxide particles surface-modified with silicone, thereby obtaining a solid component.

Here, the method may further include a step of removing impurities from the stirred solution before the stirring. In order to remove the impurities, a centrifuge, and the like, can be used, and there is no particular limitations on the method for removing the impurities.

The drying temperature may be 30 to 150° C., and more preferably 50 to 100° C.; and the drying time may be 1 to 24 hours, and more preferably 2 to 12 hours.

Further, the method for preparing a sunscreen composition according to an aspect of the present invention includes mixing in purified water the cerium oxide particles surface-modified with silicone and one or more substances selected from a group consisting of silicone oil, fibers, emulsifiers, moisturizers, and plasticizers.

The step is performed in order to mix the cerium oxide particles surface-modified with silicone with various mixtures for preparation of a cosmetic composition, thereby resulting in a formulation that is usable as a cosmetic composition.

Here, the kinds, contents, and characteristics of the silicone oils, fibers, emulsifiers, moisturizers, plasticizers that may be employed are the same as described above.

### Production Example 1: Production 1 of Cerium Oxide Particles

Cerium oxide particles were produced to prepare the sunscreen compositions according to the present invention. Cerium hydroxide, cerium carbonate, cerium nitrate, cerium chloride, and ammonium cerium nitrate may be used individually or blended and mixed. Heat treatment was performed on cerium carbonate at 500 to 1200° C. for 12 hours to obtain cerium oxide particles. The cerium oxide particles were then mixed at a 1:1 ratio with zirconia beads and ground in a bead mill for 2 hours to obtain cerium oxide particles at a size of 130 nm.

### Production Example 2: Production 2 of Calcium-Cerium Oxide Particles

Calcium-cerium oxide particles were produced to prepare the sunscreen compositions according to the present invention. Cerium hydroxide, cerium carbonate, cerium nitrate, cerium chloride, and ammonium cerium nitrate may be used individually or blended and mixed. Calcium carbonate, as the calcium salt corresponding to the cerium salt, was added at a ratio of 0.1:99.9 based on the weight percentage of calcium to cerium, and heat treatment was performed on the cerium carbonate-calcium carbonate mixture at 500 to 1200° C. for 12 hours to obtain calcium-cerium oxide particles. The calcium-cerium oxide particles were then mixed at a 1:1 ratio with zirconia beads and ground in a bead mill for 1.5 hours to obtain calcium-cerium oxide particles (0.1Ca:99.9Ce) at a size of 200 nm.

### Example 1: Preparation 1 of Sunscreen Composition According to the Present Invention

A sunscreen composition was prepared using cerium oxide particles surface-modified with triethoxycaprylylsilane.

Step 1: Surface-Modification of Cerium Oxide

First, cerium oxide particles (62.5 g, 5 wt %) having a particle size of 130 nm produced in Production Example 1 were added to and dispersed in 1,250 g of heptane, and triethoxycaprylylsilane (0.5 wt %) was added thereto.

Next, the solution was stirred at room temperature for 6 hours to coat the cerium oxide particles with triethoxycaprylylsilane. After the stirring was completed, the coated cerium oxide particles were washed three times with distilled water and the solvent was removed with an evaporator to obtain surface-modified cerium oxide particles (63 g) in the form of a solid powder.

Step 2: Preparation of a Sunscreen Composition

The surface-modified cerium oxide solid (4 wt %) obtained in step 1 above, silicone oil (DC 245, 10 wt %), fiber (VGL silk, 10 wt %), PEG silicone emulsifier (KF 6017, 3 wt %), non-ionic W/O emulsifier (Abil em 90, 2.5 wt %), moisturizer (1,2-hexanediol, 2 wt %), and purified water (68.5 wt %) were mixed together to prepare a sunscreen composition.

### Example 2: Preparation 2 of a Sunscreen Composition According to the Present Invention

A sunscreen composition was prepared using calcium-cerium oxide particles surface-modified with triethoxycaprylylsilane.

Step 1: Surface-Modification of Cerium Oxide

First, calcium-cerium oxide particles (32.25 g, 5 wt %) produced in Production Example 2 and having a particle size of 200 nm were added to and dispersed in 665 g of heptane, and triethoxycaprylylsilane (3.325 g, 0.5 wt %) was added thereto.

Next, the solution was stirred at room temperature for 6 hours to coat the calcium-cerium oxide particles with triethoxycaprylylsilane. After the stirring was completed, the coated calcium-cerium oxide particles were washed three times with distilled water and the solvent was removed with an evaporator to obtain surface-modified calcium-cerium oxide particles (29 g) in the form of a solid powder.

Step 2: Preparation of Sunscreen Composition

The surface-modified calcium-cerium oxide solid (6 wt %) obtained in step 1 above, silicone oil (DC 245, 10 wt %), fiber (VGL silk, 10 wt %), PEG silicone emulsifier (KF 6017, 3 wt %), non-ionic W/O emulsifier (Abil em 90, 2.5 wt %), moisturizer (1,2-hexanediol, 2 wt %), and purified water (66.5 wt %) were mixed together to prepare a sunscreen composition.

### Comparative Example 1: Preparation 1 of a Sunscreen Composition

A sunscreen composition was prepared using cerium oxide particles that were not surface-modified.

Cerium oxide solid (3 wt %), silicone oil (DC 245, 10 wt %), fiber (VGL silk, 10 wt %), PEG silicone emulsifier (KF 6017, 3 wt %), non-ionic W/O emulsifier (Abil em 90, 2.5 wt %), moisturizer (1,2-hexanediol, 2 wt %), and purified water (69.5 wt %) were mixed together to prepare a sunscreen composition.

### Comparative Example 2: Preparation 2 of a Sunscreen Composition

A sunscreen composition was prepared using titanium dioxide coated with silica and alumina. Titanium dioxide solid coated with silica and alumina (MT100TV manufactured by Tayca Corporation, 3 wt %), silicone oil (DC 245, 10 wt %), fiber (VGL silk, 10 wt %), PEG silicone emulsifier (KF 6017, 3 wt %), non-ionic W/O emulsifier (Abil em 90, 2.5 wt %), moisturizer (1,2-hexanediol, 2 wt %), and purified water (69.5 wt %) were mixed together to prepare a sunscreen composition.

### Comparative Example 3: Preparation 3 of a Sunscreen Composition

A sunscreen composition was prepared using zinc oxide.

Zinc oxide solid (ZnO-NAS manufactured by SUNJIN Beauty Science Co., LTD., 5 wt %), silicone oil (DC 245, 10 wt %), fiber (VGL silk, 10 wt %), PEG silicone emulsifier (KF 6017, 3 wt %), non-ionic W/O emulsifier (Abil em 90, 2.5 wt %), moisturizer (1,2-hexanediol, 2 wt %), and purified water (67.5 wt %) were mixed together to prepare a sunscreen composition.

The composition ratios of the sunscreen compositions prepared in Examples 1 and 2 and Comparative Examples 1, 2 and 3 are summarized in Table 1 below.

TABLE 1

| | Composition ratio (wt %) | | | | |
|---|---|---|---|---|---|
| | | | Comparative | Comparative | Comparative |
| Components | Example 1 | Example 2 | Example 1 | Example 2 | Example 3 |
| DC 245 | 10 | 10 | 10 | 10 | 10 |
| VGL silk | 10 | 10 | 10 | 10 | 10 |
| KF 6017 | 3 | 3 | 3 | 3 | 3 |
| Abil em 9 | 2.5 | 2.5 | 2.5 | 2.5 | 2.5 |
| 1,2-Hexanediol | 2 | 2 | 2 | 2 | 2 |
| Water | 68.5 | 66.5 | 69.5 | 69.5 | 67.5 |
| Cerium oxide particles | 4 | 6 | — | — | — |
| surface-treated with | | | | | |
| triethoxycaprylylsilane | | | | | |
| Cerium oxide particles | — | — | 3 | — | — |
| without surface modification | | | | | |
| Titanium dioxide particles | — | — | — | 3 | — |
| coated with silica and alumina | | | | | |
| Zinc oxide particles | — | — | — | — | 5 |
| Particle size | 130 nm | 200 nm | 130 nm | 130 nm | 200 nm |
| Sum | 100 | 100 | 100 | 100 | 100 |

### Experimental Example 1: Evaluation of Feeling of Use and White Cast of the Sunscreen Composition

Experiments were conducted to compare the feeling of use and the white cast of the sunscreen compositions prepared in Examples 1 and 2 and Comparative Examples 2 and 3. Comparative Example 1 was excluded from the test subjects since it was neither possible to emulsify the cerium oxide particles nor to prepare the composition into a cosmetic formulation.

Specifically, general feeling of use was evaluated on a scale of good-normal-poor in consideration of the spreadability, stability of the formulation, and the like, after application of each of the sunscreen compositions prepared in Examples 1 and 2, and Comparative Examples 2 and 3, in an amount of 0.5 cc onto the skin once. In addition, the degree of white cast was also evaluated visually and classified as being good, normal, or poor after rubbing each of the sunscreen compositions prepared in Examples 1 and 2, and Comparative Examples 2 and 3, in an amount of 0.5 cc onto the skin several times to be uniformly spread. Evaluation results of the above two experiments are shown in Table 2 below.

TABLE 2

| | | | Comparative | Comparative |
|---|---|---|---|---|
| | Example 1 | Example 2 | Example 2 | Example 3 |
| Feeling of use | Good | Good | Poor | Normal |
| White cast | Good | Good | Poor | Normal |

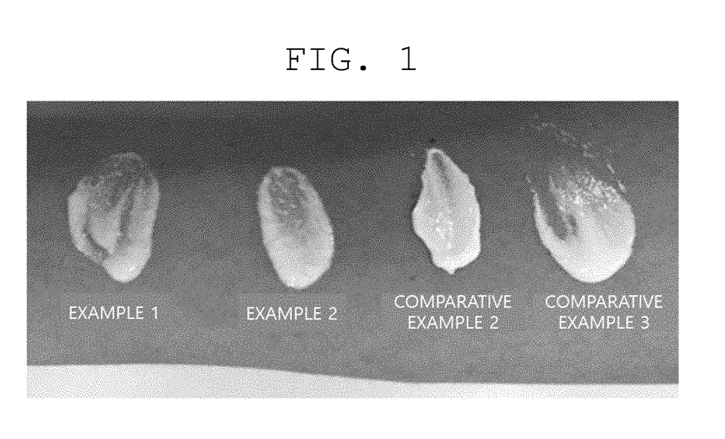

FIG. 1 shows images obtained after applying each of the sunscreen compositions prepared in Example 1, Example 2, Comparative Example 2, and Comparative Example 3 onto the skin once.

As can be seen in FIG. 1, it may be appreciated that the sunscreen compositions prepared in Examples 1 and 2 display thinner and more uniform application onto the skin as compared with Comparative Examples 2 and 3. Further, it can be confirmed in view of the color tone of the compositions that the compositions of Examples 1 and 2 exhibit relatively skin-like color in comparison to Comparative Examples 2 and 3.

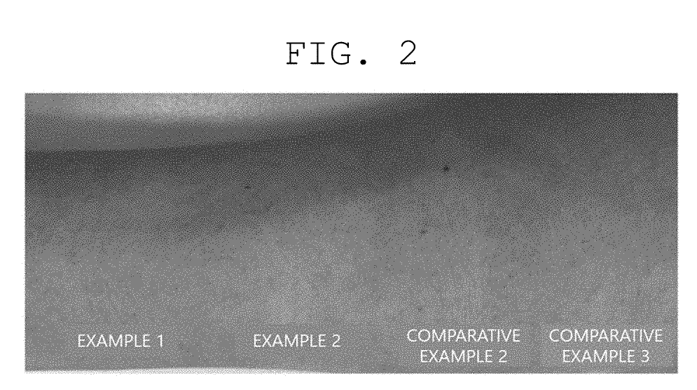

FIG. 2 shows images obtained after rubbing each of the sunscreen compositions prepared in Example 1, Example 2, Comparative Example 2, and Comparative Example 3 onto the skin several times to be uniformly spread.

As can be seen in FIG. 2, it may be appreciated that the sunscreen compositions prepared in Example 1 and Example 2 exhibit the same color as the skin and do not have a white cast. On the other hand, it may be confirmed in Comparative Example 2 and Comparative Example 3 that portions with white color occur, indicating the appearance of white cast.

Therefore, the sunscreen composition according to the present invention have excellent usability, such as excellent skin application property, spreadability, and the like, and do not cause the white cast when applied uniformly onto the skin. Therefore, the sunscreen compositions can be effectively used as compositions for producing cosmetics for UV protection.

### Experimental Example 2: Measurement of Sun Protection Factor (SPF) Spectrum of the Sunscreen Compositions

Experiments were conducted to measure and compare the SPF spectra of the sunscreen compositions prepared in Examples 1 and 2, Comparative Examples 2 and 3. Comparative Example 1 was excluded from the test subjects since it was neither possible to emulsify the cerium oxide particles nor to prepare the composition into a cosmetic formulation.

Specifically, the SPF indexes of each of the sunscreen compositions prepared in the Example 1, Example 2, Comparative Example 2 and Comparative Example 3 were measured six times, respectively, using an SPF analyzer 290S instrument manufactured by Laser Components, UK.

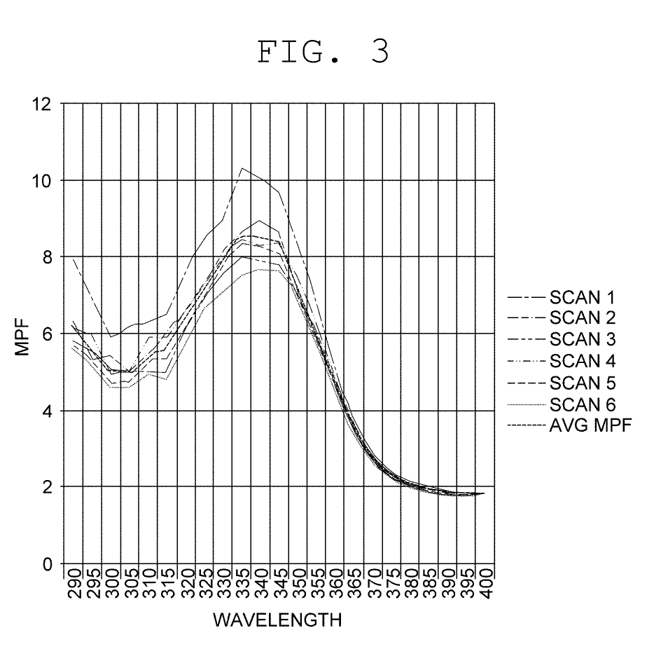

FIGS. 3 to 6 are graphs showing the measurement values of the sun protection factor (SPF) spectra of the sunscreen compositions prepared in Examples 1 and 2, and Comparative Examples 2 and 3, respectively.

As can be seen from FIG. 3, it may be appreciated that Example 1 exhibits about 8 SPF in the wavelength region of about 340 to 350.

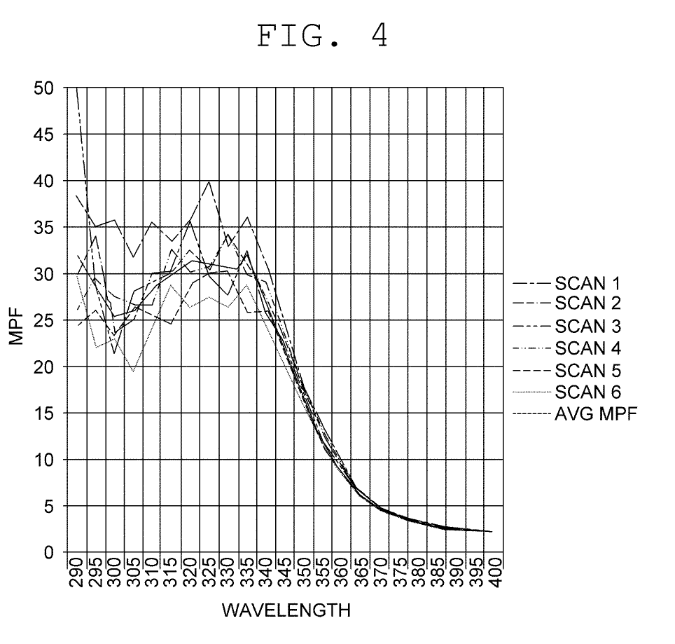

As can be seen from FIG. 4, it may be appreciated that Example 2 exhibits about 30 SPF in the wavelength region of about 315 to 340.

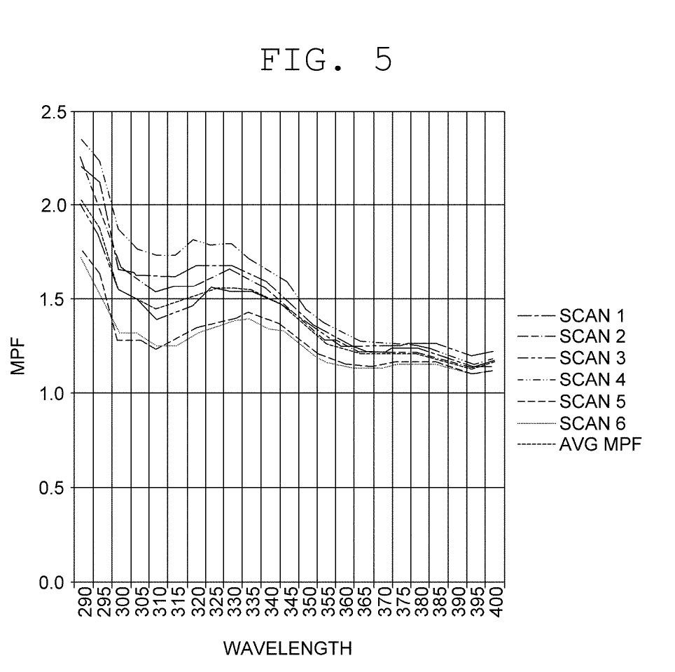

As can be seen from FIG. 5, it may be appreciated that Comparative Example 2 exhibits about 1.5 SPF in the wavelength region of about 280 to 350.

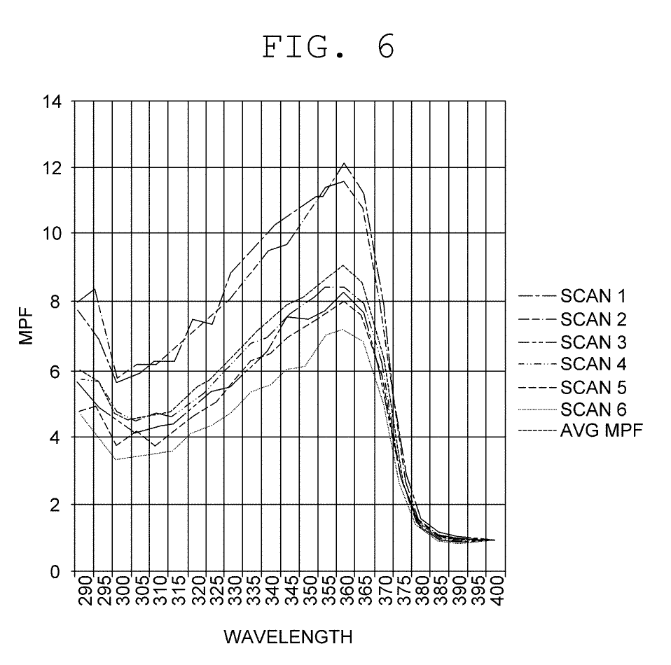

As can be seen from FIG. 6, it may be appreciated that Comparative Example 3 exhibits about 8 SPF in the wavelength region of about 345 to 365.

Therefore, the sunscreen composition according to an embodiment of the present invention effectively blocks the ultraviolet ray region, and in particular, blocks a wide range of wavelengths including not only the UVB region affecting occurrence of sunburn, but also the UVA region affecting aging, thereby allowing useful utilization as a composition for producing sunscreen cosmetics.

The sunscreen composition according to an embodiment of the present invention is easy to be prepared into a formulation applicable to cosmetics and may have a high physical ultraviolet protection effect to block ultraviolet rays in a wide range of wavelengths with an excellent sun protection factor (SPF).

In addition, the sunscreen composition according to an embodiment of the present invention has high stability of formulation, relieves irritation to the skin, and has excellent feeling of use such as texture, spreadability, and the like.

Further, the sunscreen composition according to an embodiment of the present invention has a skin-like color in view of the color tone of the composition and does not cause white cast even when applied to the skin, and thus the composition can be effectively used in producing cosmetics for UV protection that aim to express natural color.

What is claimed is:

1. A sunscreen composition comprising:
cerium oxide (CeO₂) particles that are surface-modified with silicone,
wherein the silicone is methicone, a methicone derivative, or an alkyl silane.

2. The sunscreen composition of claim 1, wherein the methicone and methicone derivative have a degree of polymerization of 3 to 50.

3. The sunscreen composition of claim 1, wherein the silicone is dimethicone, hydrogen dimethicone, triethoxysilylethyl polydimethylsiloxyethyl hexyl dimethicone, dimethicone copolyol, dimethicone crosspolymer, cyclomethicone, trimethicone, amodimethicone, dimethiconol, cyclomethicone, phenyl trimethicone, cetyl dimethicone, methylphenyl polysiloxane, cyclohexasiloxane, cyclomethasiloxane, or triethoxycaprylylsilane.

4. The sunscreen composition of claim 1, wherein the silicone is a compound represented by Chemical Formula 1 below: 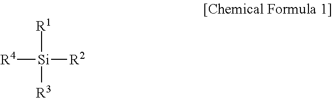
in Chemical Formula 1,
R¹, R², R³ and R⁴ are each, independently: hydrogen; a halogen; a linear or branched C₁-C₁₂ alkyl; a linear or branched C₁-C₁₂ alkyl substituted with one or more substituents selected from the group consisting of an amide group, a hydroxyl group, and a halogen; a linear or branched C₁-C₁₂ alkoxy; a linear or branched C₁-C₁₂ alkoxy substituted with one or more substituents selected from a group consisting of an amide group, a hydroxyl group, and a halogen; a C₃-C₁₂ cycloalkyl; or a C₃-C₁₂ cycloalkyl substituted with one or more substituents selected from a group consisting of an amide group, a hydroxyl group, and a halogen, provided that R¹, R², R³ and R⁴ are not all hydrogen.

5. The sunscreen composition of claim 1, wherein a coating density of the surface modification is 10 to 100% based on a surface area of the cerium oxide particle.

6. The sunscreen composition of claim 1, wherein the cerium oxide particle is doped with one or more selected from a group consisting of Al, Ti, Zn, Zr and Ca.

7. The sunscreen composition of claim 6, wherein a doping amount is 0.1 to 10 wt % based on the cerium oxide particle.

8. The sunscreen composition of claim 1, wherein the cerium oxide particle has a particle size of 0.05 to 1 μm.

9. The sunscreen composition of claim 1, wherein a content of the cerium oxide particles that are surface-modified with silicone is 5 to 50 wt % based on the total sunscreen composition.

10. A method for preparing a sunscreen composition, comprising:
adding cerium oxide particles and silicone to an organic solvent and stirring an obtained solution;
drying the stirred solution to obtain cerium oxide particles that are surface-modified with silicone; and
mixing purified water with the cerium oxide particles surface-modified with silicone and one or more substances selected from a group consisting of silicone oil, fibers, emulsifiers, moisturizers, and plasticizers.
